(19) United States
(12) Patent Application Publication
(10) Pub. No.: US 2014/0357723 A1
(43) Pub. Date: Dec. 4, 2014

(54) Tricholine Nasal Formulation and Method of Use

(71) Applicant: Uwe-Bernd Bernd Ross, Koenigstein (DE)

(72) Inventors: Christiane Voss, Kronberg (DE); Uwe-Bernd Rose, Koenigstein (DE)

(21) Appl. No.: 13/904,552

(22) Filed: May 29, 2013

(51) Int. Cl. A61K 47/46; A61K 31/194; A61K 47/02; A61K 47/10; A61K 31/14; A61K 47/36

(52) U.S. Cl. CPC A61K 47/46; A61K 31/14; A61K 47/36; A61K 47/02; A61K 47/10; A61K 31/194
USPC 514/642

(57) ABSTRACT

A commercially-suitable tricholine nasal formulation for administration to a human as a nasal spray is disclosed. The tricholine nasal formulation includes an aqueous solution of tricholine citrate, a volatile oil and a buffer system. The disclosed tricholine nasal formulation possesses an acceptable odor and a reasonable shelf-life. The tricholine nasal formulation may be used to treat the symptoms associated with stress, anxiety, depression, malaise, or “burn out” syndrome. Optionally, a moisturizer is included in the composition to alleviate any dryness resulting from administration of the formulation to a user. A preservative may be included in the composition to produce a longer shelf-life.

### FIELD OF THE DISCLOSURE

The subject matter of the present disclosure generally relates to choline supplement products, and more particularly to a topically delivered tricholine citrate formulation for nasal administration.

### BACKGROUND OF THE DISCLOSURE

The use of choline products to relieve stress, anxiety, depression, malaise, or “burn out” syndrome has been previously disclosed. Historically, these products were administered as injections, as oral administration is precluded by the metabolic profile of choline. Choline injectable products were previously marketed under the brand name Neurotropan. To be effective, the injections required regularly scheduled administration by a health care professional. Thus, patients' schedules had to be interrupted to allow for frequent doctor visits. For this and other reasons, choline injection products ceased to be manufactured and available to patients and physicians.

The delivery of active ingredients in the form of nasal formulations has also been previously disclosed. However, preparation of a nasal formulation of choline presented various obstacles. First, an effective concentration of choline would need to be nasally administered. Second, a choline nasal product would need to have a commercially reasonable shelf-life. Third, the choline nasal product would need to be non-irritating. Fourth, the choline nasal product would need to be pleasing to the patient, i.e. the formulation should have a neutral or pleasant odor.

Tricholine citrate is a preferred choline source in a nasal formulation for various reasons. First, the tricholine salt provides a higher choline concentration on a weight/weight basis than other choline salts. The increased choline concentration allows for the delivery of comparatively larger quantity of choline per a similarly sized dose than other choline sources. Second, tricholine citrate is also readily soluble in water. Third, tricholine citrate is readily available from multiple commercial sources. However, the use of tricholine citrate in a nasal formulation presents several problems.

First, tricholine citrate has an odor that is considered repugnant, and therefore it would not readily appear viable as a candidate for delivery via nasal passageways, in which a substance's odor is of particular importance. Second, administration of a tricholine citrate aqueous solution may result in irritation to the nasal passageways and mucous membranes of a human, thereby creating discomfort for potential users. Third, an aqueous solution of tricholine citrate is unstable and will degrade relatively quickly, and result in an unacceptably short shelf-life and would not be considered commercially viable as a consumer product.

The subject matter of the present disclosure is directed to overcoming, or at least reducing the effects of, one or more of the problems set forth above.

### BRIEF SUMMARY OF THE INVENTION

The disclosed invention is a commercially-suitable choline nasal formulation that may be administered to humans. The composition includes an aqueous solution of tricholine citrate, a volatile oil and a buffer system. The buffer system serves to stabilize the compound while the volatile oil improves the odor of the composition. Optionally, a moisturizer is included in the composition to counter irritation from dryness that may result from administration of the composition to a user.

The disclosed tricholine nasal formulation has several advantages. First, the tricholine nasal formulation administered in a nasal spray eliminates the need for users to make frequent doctor visits for injections. Second, the tricholine nasal formulation is less irritating to the user's nasal passageways and mucous membranes, while still allowing for the effective administration of choline. The use of a buffer system reduces tricholine degradation and results in a commercially acceptable shelf-life for the product. Also, controlling the pH of the formulation within the disclosed range reduces nasal irritation. Other benefits of the disclosed subject matter may also be apparent to those in the art to which the disclosure pertains. Collectively, these attributes make the disclosed subject matter commercially viable as a product.

The details of one or more embodiments of the invention are set forth in the accompanying descriptions below. The foregoing summary is not intended to summarize each potential embodiment or every aspect of the disclosure.

### DETAILED DESCRIPTION OF THE DISCLOSURE

Disclosed is a tricholine nasal formulation, the composition of which overcomes several problems apparent in previously disclosed formulations and methods of delivery of choline products. The delivery of a choline product as a nasal spray is advantageous in that users can be given a nasal sprayer to use on their own, as opposed to requiring users to visit a doctor on a weekly or otherwise regular basis to receive injections. Therefore, cost and inconvenience for the user are reduced, making the tricholine nasal formulation better suited for the treatment of patients.

A basic embodiment composition includes an aqueous solution of tricholine citrate, a volatile oil, and buffer system in a concentration sufficient to stabilize the composition. Various combinations of chemical compounds may serve as buffer systems and are known in the art. The volatile oil serves to at least partially, or ideally completely, improve or mask the odor of the overall composition when the composition is administered in an effective dosage to a user. Various volatile oils are suitable for this purpose and for use in conjunction with the disclosed subject matter. Optionally, such a volatile oil could be selected from among those approved for topical use as known in the art.

This basic embodiment may cause some dryness irritation in the nasal cavity and mucous membranes of users. Therefore, it may be advantageous to include a moisturizer in the tricholine nasal formulation such that irritation due to dryness may be prevented or remedied. Optionally, such a moisturizer could be selected from among those already approved for topical use. A moisturizer may include a water thickener to aid in the retention of the composition at the site of application.

It may also be advantageous to include a preservative in the tricholine nasal formulation to enhance the longevity of the composition. For instance, phenoxyethanol may be used for this purpose.

In a certain embodiment, a tricholine nasal formulation includes the following constituents: first, an aqueous solution of tricholine citrate in a concentration inclusive of 10 to 65 percent by weight; second, a buffer system designed to maintain the pH of the tricholine nasal formulation between 7.0 and 7.4 (a preferred buffer system includes sodium chloride, calcium chloride, disodium hydrogen phosphate and calcium hydrogen phosphate, in a concentration sufficient to maintain the pH of the composition at a value inclusive of 7.3 to 7.4); third, a moisturizer (hyaluronic acid or any methyl cellulose derivative), in a concentration sufficient to reduce irritation caused by dryness when the tricholine nasal formulation is administered in an effective dose; and lastly, a volatile oil (e.g., oil of citrus or oil of lavender) is used, in a concentration sufficient to substantially improve the odor of the composition of when the composition is administered in a tricholine nasal formulation.

In a specific demonstrated embodiment (Example 1), the above composition has the following relatively weighted values: tricholine citrate in a weight of 1.6 g, hyaluronic acid in a weight of 10 mg, sodium chloride in a weight of 80 mg, calcium chloride in a weight of 2 mg, disodium hydrogen phosphate in a weight of 11.5 mg, calcium hydrogen phosphate in a weight of 2 mg, and oil of citrus in a weight of 200 mg.

### EXAMPLE 1

| | | |
|---|---|---|
| Tricholine citrate | 1.6 | g |
| hyaluronic acid | 10 | mg |
| sodium chloride | 80 | mg |
| calcium chloride | 2 | mg |
| disodium hydrogen phosphate | 11.5 | mg |
| calcium hydrogen phosphate | 2 | mg |
| oil of citrus | 200 | mg |

| | |
|---|---|
| Distilled water | q.s. 5 ml |

As previously noted, the disclosed tricholine citrate composition is suitable for delivery by a nasal sprayer. There are various sprayers, many of which are used for delivering other medications or chemicals that would be expected to suffice for administration of the disclosed composition.

It is noted that an effective amount of an embodiment of the disclosed composition may be any amount sufficient to produce the desired effect on the user of the nasal spray. Alternatively, an effective amount may be defined as some amount of the composition that is found to be effective in a particularly large subset of users. The administration of an effective amount of the disclosed composition may require multiple sprays from a nasal sprayer, the number of which may depend on such factors as the desired dosage, the geometry or capability of the nasal sprayer, the user's typical affinity or lack thereof for the composition, or other factors.

The foregoing description of preferred and other embodiments is not intended to limit or restrict the scope or applicability of the inventive concepts conceived of by the Applicants. In exchange for disclosing the inventive concepts contained herein, the Applicants desire all patent rights afforded by the appended claims. Therefore, it is intended that the appended claims include all modifications and alterations to the full extent that they come within the scope of the following claims or equivalents thereof.

What is claimed is:

1. A tricholine citrate nasal spray composition, comprising:
an aqueous solution of tricholine citrate;
a volatile oil; and
a buffer system in a concentration sufficient to stabilize the composition.

2. The composition of claim 1, wherein the tricholine citrate is in said aqueous solution in a concentration inclusive of 10 to 65 percent by weight.

3. The composition of claim 1, wherein said buffer system is in a concentration sufficient to adjust the pH of the composition to a value inclusive of 7.0 to 7.8.

4. The composition of claim 1, further comprising a moisturizer.

5. The composition of claim 4, wherein said moisturizer is in a concentration sufficient to substantially reduce irritation caused by dryness when said composition is administered in an effective amount as a nasal spray.

6. The composition of claim 4, wherein said moisturizer is hyaluronic acid.

7. The composition of claim 1, wherein said buffer system comprises sodium chloride, calcium chloride, disodium hydrogen phosphate and calcium hydrogen phosphate.

8. The composition of claim 1, wherein said volatile oil is in a concentration sufficient to substantially improve the odor of said composition when said composition is administered in an effective amount as a nasal spray.

9. The composition of claim 1, wherein said volatile oil is oil of citrus.

10. The composition of claim 1, further comprising a preservative.

11. The composition of claim 10 wherein said preservative is phenoxyethanol.

12. The composition of claim 5, wherein:
said composition further includes a preservative;
said preservative is phenoxyethanol;
said moisturizer is hyaluronic acid;
said buffer system comprises sodium chloride, calcium chloride, disodium hydrogen phosphate and calcium hydrogen phosphate and is in a concentration sufficient to adjust the pH of the composition to a value inclusive of 7.3 to 7.4; and
said volatile oil is oil of citrus and is in a concentration sufficient to substantially improve the odor of said composition when said administering step is performed.

13. A method of topically administering a tricholine citrate composition, comprising the steps of:
providing a nasal sprayer containing an amount of said tricholine citrate composition, said tricholine citrate composition including an aqueous solution of tricholine citrate, a volatile oil and a buffer system in a concentration sufficient to stabilize the composition; and
administering using said nasal sprayer an effective amount of said tricholine citrate composition.

14. The method of claim 13, wherein the tricholine citrate is in said aqueous solution in a concentration inclusive of 10 to 65 percent by weight.

15. The method of claim 13, wherein said buffer system is in a concentration sufficient to adjust the pH of the composition to a value inclusive of 7.0 to 7.8.

16. The method of claim 13, wherein said tricholine cirtrate composition further comprises a moisturizer.

17. The method of claim 16, wherein said moisturizer is in a concentration sufficient to substantially reduce irritation caused by dryness after said administering step is performed.

18. The method of claim 16, wherein said moisturizer is hyaluronic acid.

19. The method of claim 13, wherein said buffer system comprises sodium chloride, calcium chloride, disodium hydrogen phosphate and calcium hydrogen phosphate.

20. The method of claim 13, wherein said volatile oil is in a concentration sufficient to substantially improve the odor of said composition when said administering step is performed.

21. The method of claim 13, wherein said volatile oil is oil of citrus.

22. The method of claim 13, further comprising a preservative.

23. The method of claim 22, wherein said preservative is phenoxyethanol.

24. The method of claim 17, wherein:
said tricholine citrate composition further includes a preservative;
said preservative is phenoxyethanol;
said moisturizer is hyaluronic acid;
said buffer system comprises sodium chloride, calcium chloride, disodium hydrogen phosphate and calcium hydrogen phosphate and is in a concentration sufficient to adjust the pH of the composition to a value inclusive of 7.3 to 7.4; and
said volatile oil is oil of citrus and is in a concentration sufficient to substantially improve the odor of said composition when said administering step is performed.
